(19) United States
(12) Patent Application Publication
(10) Pub. No.: US 2019/0133694 A1
(43) Pub. Date: May 9, 2019

(54) SURGICAL TRACKING SYSTEM FOR TRACKING AND VISUALIZING THE RELATIVE POSITIONING OF TWO OR MORE SURGICAL COMPONENTS

(71) Applicant: COVIDIEN LP, Mansfield, MA (US)

(72) Inventors: OSVALDO ANDRES BARRERA, MADISON, CT (US); SHUTING GUO, SAN FRANCISCO, CA (US)

(21) Appl. No.: 16/177,544

(22) Filed: Nov. 1, 2018

Related U.S. Application Data
(60) Provisional application No. 62/580,893, filed on Nov. 2, 2017.

(51) Int. Cl. A61B 34/20; A61B 34/30; A61B 34/00; A61B 90/00; A61B 34/10

(52) U.S. Cl. CPC A61B 34/20; A61B 34/30; A61B 34/25; A61B 90/37; A61B 34/10; A61B 90/361; A61B 2090/364; A61B 2034/305; A61B 2034/2063; A61B 2034/2055; A61B 2034/2051

(57) ABSTRACT

A surgical visualization and tracking system includes including first and second tracking magnets and a sensor base assembly including a base and at least three magnetic sensor assemblies. Each of the magnetic sensor assemblies is configured to independently track each of the tracking magnets in X, Y, and Z dimensions. A control console is configured to receive tracking data from each of the magnetic sensor assemblies and determine position and orientation information for each of the tracking magnets based upon the received tracking data. A visualization system is configured to receive the position and orientation information from the control console and represent the position and orientation information for each of the first and second tracking magnets on a displayed 3D model of tissue.

### CROSS-REFERENCE TO RELATED APPLICATIONS

This application claims the benefit of, and priority to, U.S. Provisional Patent Application No. 62/580,893, filed on Nov. 2, 2017, the entire contents of which are hereby incorporated herein by reference.

### BACKGROUND

### Technical Field

The present disclosure relates to a surgical tracking system for tracking and visualizing the relative positioning of two or more surgical components, e.g., an implanted surgical marker and an end effector of a surgical instrument, within a surgical site during a surgical procedure.

### Background of Related Art

Pre-implanted magnetic markers are utilized in some surgical procedures to facilitate tracking. However, the use of magnetic markers has some drawbacks at least because a patient with an implanted magnetic marker cannot undergo an MRI procedure due the incompatible nature of an implanted magnetic marker and an MRI machine. Magnetic markers may also interfere with other imaging systems such as ultrasound imaging systems and X-ray imaging systems.

Guidewires extending from implanted markers are also utilized in surgical procedures. However, the use of guidewires has drawbacks at least because of patient discomfort and migration problems.

Direction-only guidance technology is utilized in surgical procedures but, as the name suggests, does not provide information beyond the distance and direction to an implanted marker.

Optical systems for surgical navigation also exist but suffer from the drawback that a direct line of sight needs to be maintained between the sensor and sensed object.

Other commercial tracking systems utilize electromagnetic sensors and tracking seeds, but such tracking systems have only one dimensional tracking capability, and are only able to provide linear distance indications. Traditional surgical navigation systems also have drawbacks in that they are not compatible with some implantable markers.

Accordingly, there is a continuing need for a surgical tracking system for tracking and visualizing the relative positioning of two or more surgical components within a surgical site during a surgical procedure.

### SUMMARY

The present disclosure provides a surgical tracking system for tracking and visualizing the relative positioning of two or more surgical components, e.g., an implanted surgical marker and an end effector of a surgical instrument, within a surgical site during a surgical procedure. As used herein, the term “distal” refers to the portion that is being described which is further from a surgeon, while the term “proximal” refers to the portion that is being described which is closer to a surgeon. The terms “about,” substantially,” and the like, as utilized herein, are meant to account for manufacturing, material, environmental, use, and/or measurement tolerances and variations. Further, to the extent consistent, any of the aspects described herein may be used in conjunction with any or all of the other aspects described herein.

A surgical tracking system provided in accordance with the present disclosure includes a first tracking magnet, a second tracking magnet, and a sensor base assembly. The sensor base assembly includes a base defining an opening therethrough and a plurality, e.g., at least three, magnetic sensor assemblies disposed on the base and arranged about the opening. Each of the magnetic sensor assemblies is configured to independently track each of the first and second tracking magnets in X, Y, and Z dimensions.

In an aspect of the present disclosure, the surgical tracking system further includes a control console configured to receive tracking data from each of the magnetic sensor assemblies and determine position and orientation information for each of the first and second tracking magnets based upon the received tracking data.

In another aspect of the present disclosure, the sensor base assembly is configured for positioning such that at least a portion of tissue of interest extends through the opening of the base.

In still another aspect of the present disclosure, the first tracking magnet is configured for implantation into tissue of interest. In such aspects, the sensor base assembly may be positioned such that at least a portion of the tissue of interest extends through the opening of the base.

In yet another aspect of the present disclosure, the second tracking magnet is associated with a surgical instrument. The second tracking magnet, more specifically, may be incorporated into the surgical instrument or coupled (permanently or releasably) to the surgical instrument.

In still yet another aspect of the present disclosure, the base is adjustable to adjust at least one dimension of the opening of the base.

In another aspect of the present disclosure, at least four magnetic sensor assemblies are provided and radially arranged (in aspects, radially symmetrically arranged) about the base. In other aspects, at least eight magnetic sensor assemblies are provided and radially arranged (in aspects, radially symmetrically arranged) about the base.

A surgical visualization and tracking system in accordance with aspects of the present disclosure includes a surgical tracking system and a visualization system. The surgical tracking system includes first and second tracking magnets and a sensor base assembly including at least three magnetic sensor assemblies. Each magnetic sensor assembly is configured to independently track each of the first and second tracking magnets in X, Y, and Z dimensions. The surgical tracking system further includes a control console configured to receive tracking data from each of the magnetic sensor assemblies and determine position and orientation information for each of the tracking magnets based upon the received tracking data. The visualization system is configured to receive the position and orientation information from the control console and represent the position and orientation information for each of the first and second tracking magnets on a displayed 3D model of tissue.

In an aspect of the present disclosure, the sensor base assembly further includes a base defining an opening therethrough. In such aspects, the magnetic sensor assemblies are disposed on the base and arranged about the opening. The base may further be configured for positioning such that at least a portion of tissue of interest extends through the opening of the base.

In another aspect of the present disclosure, the first tracking magnet is configured for implantation into tissue of interest and the second tracking magnet is associated with a surgical instrument.

In yet another aspect of the present disclosure, at least four magnetic sensor assemblies are provided and radially (in aspects, radially symmetrically) arranged.

In still another aspect of the present disclosure, the visualization system is further configured to receive instrument information regarding a surgical instrument with which the second tracking magnet is associated. In such aspects, representing the position and orientation information for the second tracking magnet on the displayed 3D model of tissue includes representing at least a portion of the surgical instrument on the displayed 3D model of tissue.

In still yet another aspect of the present disclosure, the instrument information includes information indicating a distance from the second tracking magnet to a feature of the surgical instrument.

In another aspect of the present disclosure, the represented position and orientation information is updated in real time.

In still another aspect of the present disclosure, the visualization system includes a non-transitory computer-readable storage medium storing a visualization application that, when executed by a processor, receives the position and orientation information from the control console and represents the position and orientation information for each of the first and second tracking magnets on the displayed 3D model of tissue.

In another aspect of the present disclosure, the visualization system further includes a display including a user interface. The visualization application, when executed by the processor, outputs the representation and the displayed 3D model of tissue for display on the user interface of the display.

### BRIEF DESCRIPTION OF THE DRAWINGS

Various aspects and features of the present disclosure are described hereinbelow with reference to the drawings wherein like numerals designate identical or corresponding elements in each of the several views and:

FIG. 1 is a perspective view of an experimental set-up illustrating a surgical tracking system provided in accordance with the present disclosure;

FIG. 2 is a block diagram of the surgical tracking system of FIG. 1;

FIG. 3 is a top view of a sensor base assembly of the surgical tracking system of FIG. 1;

FIG. 4 is a side view of the sensor base assembly of FIG. 3 disposed about a patient's breast;

FIG. 5A is a top view of an adjustable sensor base assembly configured for use with the surgical tracking system of FIG. 1, disposed in a first position;

FIG. 5B is a top view of the adjustable sensor base assembly of FIG. 5A, disposed in a second position; and

FIGS. 6A-6C, 7, and 8 illustrate various different surgical instruments configured for use with the surgical tracking system of FIG. 1.

### DETAILED DESCRIPTION

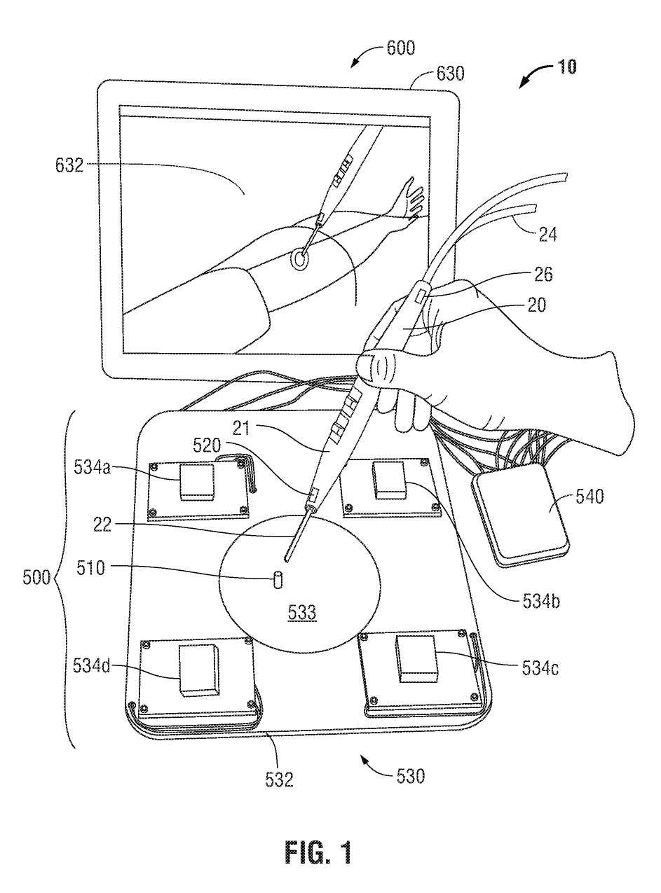

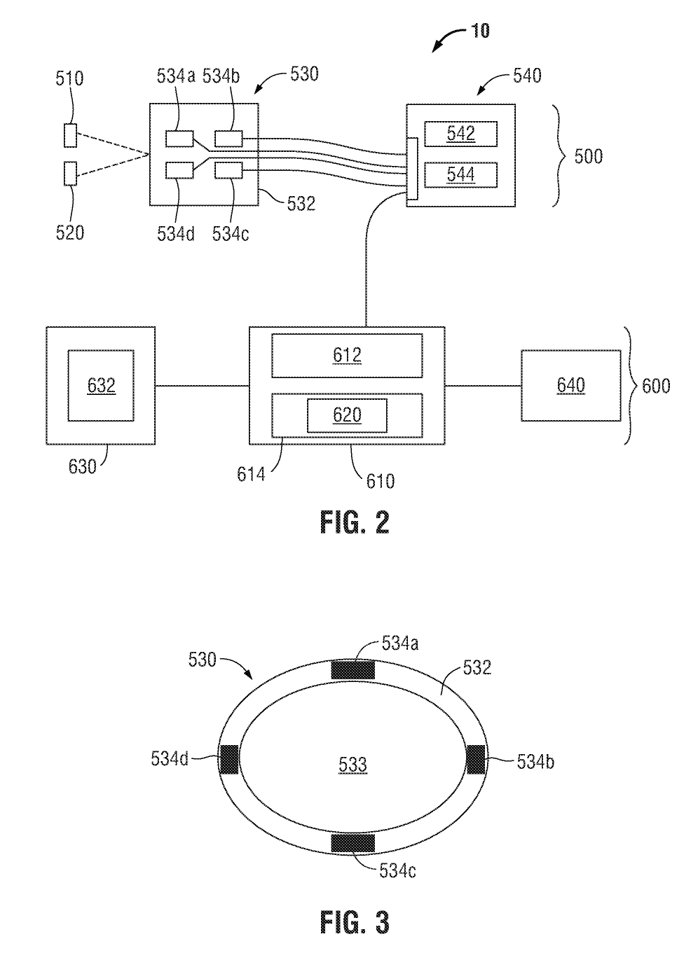

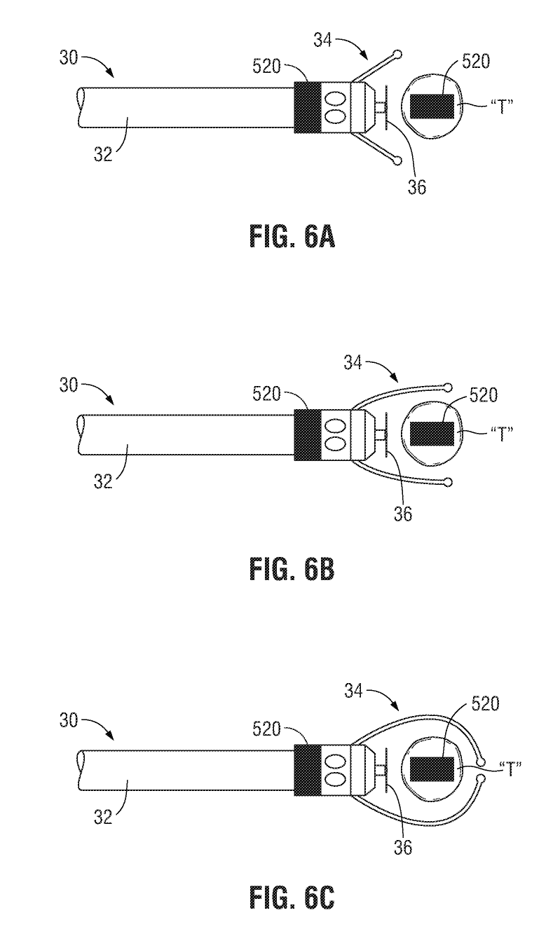

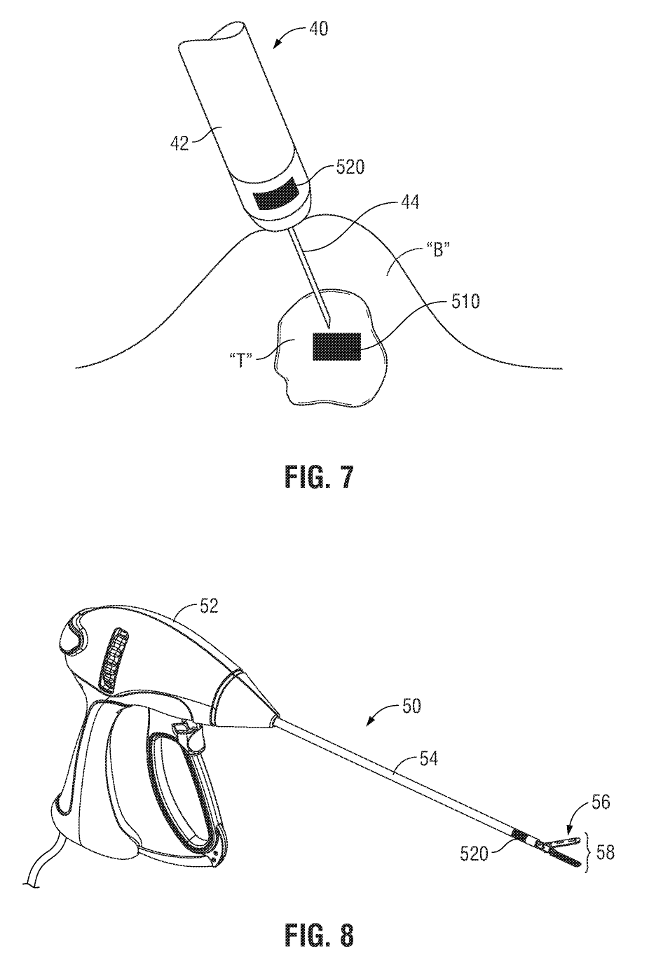

Referring to FIGS. 1 and 2, the present disclosure provides a tracking and visualization system 10 that facilitates the relative spatial location and orientation tracking between two or more surgical components such as, for example, a tissue marker disposed within a tissue of interest, e.g., a tumor “T” (see, e.g., FIGS. 6A-7), and a surgical instrument 20, 30, 40, and 50 (FIGS. 1, 6A-6C, 7, and 8, respectively), for biopsying, resecting, or otherwise performing a surgical task on the tumor “T.” System 10 includes two sub-systems: a geomagnetic tracking system 500 that enables navigation of the surgical instrument 20, 30, 40, and 50 (FIGS. 1, 6A-6C, 7, and 8, respectively) relative to the tumor “T;” and a visualization system 600 including a visualization application 620 running on a suitable computer 610 for outputting, to a user interface 632 of a display 630, a 3D virtual environment for real-time visualization of the navigation.

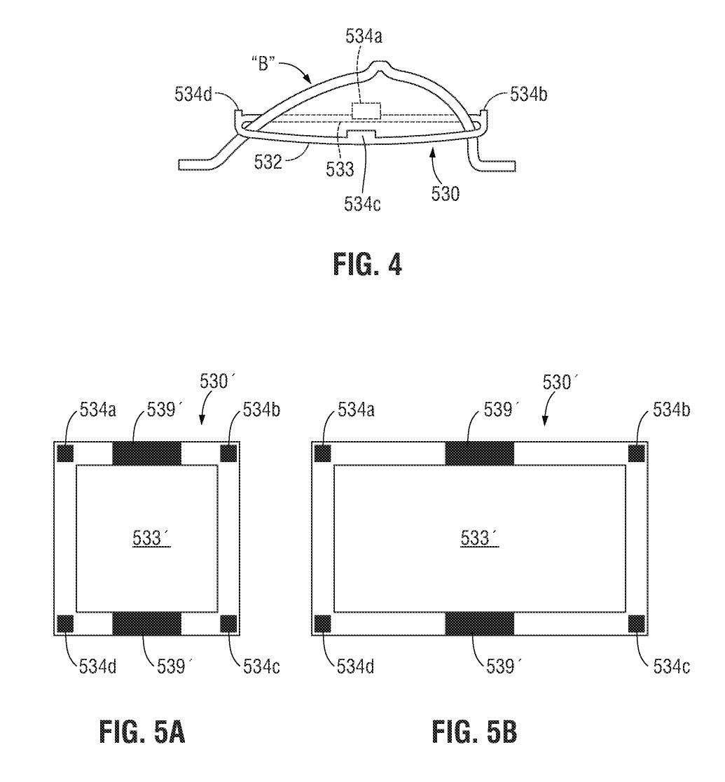

Geomagnetic tracking system 500 generally includes two or more tracking magnets 510, 520; a sensor base assembly 530 including a base 532 and three or more, e.g., four, eight, or other suitable number, magnetic sensor assemblies 534a-534d; and a control console 540. Magnetic sensor assemblies 534a-534d are configured to detect the positions of magnets 510, 520 and output position data to control console 540 which receives, as input, the position data from magnetic sensor assemblies 534a-534d and calculates the position and orientation of each magnet 510, 520 based upon the input data. Control console 540 is further configured to output the calculated position and orientation data to visualization application 620 of visualization system 600 to enable display of a 3D virtual environment for real-time guidance including, in embodiments, surface and/or volumetric models of the patient's anatomy (e.g., breast “B” (FIG. 4) and tumor “T” (FIGS. 6A-7)), surgical instruments 20, 30, 40, and 50 (FIGS. 1, 6A-6C, 7, and 8, respectively) utilized during the procedure, and/or visual aids (e.g., safe resection margins around the tumor “T” (FIGS. 6A-7), alignment axes, possible trajectories, etc.).

Continuing with reference to FIGS. 1 and 2, tracking magnets 510, 520 may be relatively small, cylindrical magnet seeds or markers. Alternatively, tracking magnets 510, 520 may be integral or removable portions of other components, e.g., implants, surgical instruments, etc. Tracking magnets 510, 520 may, in embodiments, be permanent magnets with predetermined materials, shapes, and sizes, although other configurations are also contemplated. Tracking magnets 510, 520 may be, in embodiments, rare earth magnets, e.g., neodymium magnets (Nd2Fe14B), and/or have cylindrical shapes. Tracking magnets 510, 520 have constant geomagnetic fields thus enabling sensor base assembly 530, as detailed below, to exploit the constant geomagnetic fields thereof to provide orientation and location information for each of tracking magnets 510, 520.

One or more of the tracking magnets 510, for example, may be implanted, e.g., within a tumor “T” (FIGS. 6A-7) to be removed, while at least one other tracking magnet 520 is coupled to or incorporated into a surgical instrument 20, 30, 40, and 50 (FIGS. 1, 6A-6C, 7, and 8, respectively). With respect to the tracking magnet(s) 510 implanted into the tumor “T,” implantation may be effected utilizing a suitable marking device under ultrasound guidance such as, for example, as detailed in U.S. Provisional Patent Application No. 62/647,185, titled “Surgical Assemblies Facilitating Tissue Marking and Methods of Use Thereof,” filed on Mar. 23, 2018, the entire contents of which are hereby incorporated herein by reference.

The tracking magnet(s) 510 implanted within the tumor “T” is preferably implanted near-in-time to the tumor removal procedure, thus avoiding the drawbacks of previously-implanted magnetic markers. The implanted tracking magnet(s) 510 is configured to be removed with the tumor “T” during the tumor removal procedure and, thus, the patient may undergo, for example, MRI procedures post-surgery and avoids the other drawbacks associated with an implanted magnetic marker.

The other tracking magnet(s) 520, as noted above, is coupled to or incorporated into a surgical instrument 20, 30, 40, and 50 (FIGS. 1, 6A-6C, 7, and 8, respectively). For example, the manufacturer of the surgical instrument may form a portion of the instrument from a suitable magnetic material functioning as tracking magnet 520, place tracking magnet 520 at a suitable location during assembly of the instrument, or otherwise integrally incorporate tracking magnet 520 into the instrument. Alternatively, tracking magnet 520 may be coupled to the instrument post-manufacturing such as, for example, at the end-user. Tracking magnet 520 may be coupled to the instrument in any suitable releasable or non-releasable manner such as, for example, using adhesives, tape, mechanical engagement (e.g., clips, bands, or latches), etc.

As shown in FIG. 1, for example, tracking magnet 520 is utilized with an electrosurgical pencil 20 such as, for example, described in U.S. Pat. No. 7,879,033 to Sartor et al., the entire contents of which are hereby incorporated herein by reference. Tracking magnet 520 is positioned on or within housing 21 of electrosurgical pencil 20 adjacent to blade 22 thereof in close proximity thereto without interfering with or being interfered by the function of blade 22. Various other exemplary surgical instrument 30, 40, and 50 (FIGS. 6A-6C, 7, and 8, respectively) including tracking magnet 520 are detailed below. However, these are merely exemplary as tracking magnet 520 may be utilized with any suitable surgical instrument.

In embodiments, an identifier device 26 is associated with the surgical instrument, e.g., surgical instrument 20, or the tracking magnet 520 itself. Identifier device 26 may be a memory chip or other suitable circuitry programmed or formatted to store or generate, for example, identification, type, calibration, positioning, and/or other information regarding the surgical instrument with which the tracking magnet 520 is associated. In embodiments, a connector 24 associated with the surgical instrument, e.g., surgical instrument 20, is configured to be electronically (via wired or wireless connection) coupled to visualization system 600 to relay information from identifier device 26 to visualization system 600. Alternatively, information regarding the surgical instrument may be manually input or selected at the visualization system 600.

Identifier device 26 (FIG. 1) or another suitable device may, in embodiments, record and/or store spatial location information such as, for example, a spatial location of the distal tip, end effector components or features, etc., of the instrument relative to the position of tracking magnet 520. As a result, tracking information obtained from tracking magnet 520 may be extrapolated into tracking information regarding the movement and positioning of components or features of the instrument, thus enabling 3D tracking of a portion of the instrument in real-time during a procedure. Additional or alternative navigation-related circuitry components can also be provided such as, for example an accelerometer, gyroscopic sensor, or other inertial sensor (not shown).

With additional reference to FIGS. 3-4, as noted above, sensor base assembly 530 includes a base 532 and three or more, four or more, etc., magnetic sensor assemblies 534a-534d. Base 532 may be formed from any suitable material, may define any suitable shape, e.g., oval, square, etc., and defines an interior opening 533 of any suitable size/shape, e.g., oval, square, etc. Base 532 may be substantially rigid (e.g., not intended to substantially flex during positioning), semi-rigid (e.g., configured to allow for some flexion to facilitate positioning), or flexible (e.g., configured to be flexed during positioning). As illustrated in FIG. 4, in embodiments, base 532 is positioned about a patient's breast “B” such that the breast “B” is at least partially received through interior opening 533 of base 532. Base 532 may be vertically positioned such that magnetic sensor assemblies 534a-534d disposed thereon are substantially aligned, e.g., co-planar, with the implanted tracking magnet 510, thereby minimizing magnet-to-sensors distances and, as a result, maximizing tracking accuracy. However, other configurations are also contemplated.

Magnetic sensor assemblies 534a-534d are arranged, e.g., mounted on base 532, in substantially co-planar relation relative to one another and may be distributed in a radially-symmetric pattern, e.g., at 0°, 90°, 180° and 270°, or in any other suitable manner to enable sensor assemblies 534a-534d to be utilized to triangulate the location of tracking magnets 510, 520. In embodiments, another set of four magnetic sensor assemblies (not shown) are disposed in a similar pattern on the opposite side of base 532, for example, to increase data redundancy. Each magnetic sensor assembly 534a-534d includes an arrays of three sensors (e.g., RM3100 geomagnetic sensors available from PNI Sensor Corporation of Santa Rosa, Calif., USA), to enable each magnetic sensor assembly 534a-534d to independently detect magnetic variations in the “x,” “y,” and “z” directions and, thus, to independently track tracking magnets 510, 520 in three dimensions. Magnetic sensor assemblies 534a-534d may additionally or alternatively include other electromagnetic sensors such as, for example, magnetic resistive sensors, additional marking devices, Hall-effect sensors, etc., and/or may additionally or alternatively utilize a different tracking technology, e.g., radiation, ultra wide band (UWB), RFID, etc.

Based upon the arrangement and number of magnetic sensor assemblies 534a-534d, in embodiments, magnetic sensor assemblies 534a-534d may be configured to provide at least three degrees of freedom (DOF) tracking of the location of either or both tracking magnets 510, 520, at least four DOF tracking of the location of either or both tracking magnets 510, 520, at least five DOF tracking of the location of either or both tracking magnets 510, 520 or, in still other embodiments, at least six DOF tracking of the location of either or both of tracking magnets 510, 520. In embodiments, the sixth DOF can be added with an external tracking system (not shown), e.g., a sub-degree heading unit or optical tracker system. Nine-degrees-of-freedom sensors (not shown) using, for example, magnetometers, accelerometers, gyroscopes, and/or optical stereo-trackers may also be established to provide additional DOF tracking.

In embodiments, the DOF for the tracking magnets 510, 520 may be different. For example, where orientation determination is not as critical, such as, for example, with respect to the orientation of tracking magnet 510 and/or the tumor “T” (see, e.g., FIGS. 6A-7) relative to base 532, three DOF tracking may be sufficient. Six or more DOF tracking may be utilized for the tracking magnet 520 of the surgical instrument 20 to enable determination of the relative position and orientation of the surgical instrument 20 relative to the tracking reference, e.g., base 532. In such embodiments, both the three and six or more DOF coordinate systems may be integrated to enable determination of the position and orientation (with six DOF) of the instrument 20 relative to the tumor “T.”

Magnetic sensor assemblies 534a-534d are disposed at determined or determinable locations. More specifically, magnetic sensor assemblies 534a-534d may be disposed at determined, fixed locations relative to one another, e.g., in embodiments where base 532 is rigid and defines fixed dimensions; or may be disposed at adjustable locations relative to one another that are determinable via user input, calibration, etc., e.g., in embodiments wherein base 532 is flexible or adjustable. Calibration of the relative locations of magnetic sensor assemblies 534a-534d may be provided using an infrared (IR) array incorporated into magnetic sensor assemblies 534a-534d to enable self-calibration, or via any other suitable calibration method or device (incorporated into magnetic sensor assemblies 534a-534d or separately therefrom). The relative locations of magnetic sensor assemblies 534a-534d, whether pre-determined or subsequently determined, are input to control console 540 to enable accurate tracking.

Referring momentarily to FIGS. 5A and 5B, another embodiment of a sensor base assembly 530′, having an adjustable configuration, is provided. More specifically, base 532′ may include adjustment mechanisms 539′, e.g., incremental ratchet mechanisms, incremental snap-fit engagements, continuous friction-fit engagements, etc., to enable adjustable such as, for example, to vary at least one dimension of interior opening 533′ to facilitate positioning of base 532′ about different patient anatomies, to enable use on various different patient body types or conditions, etc.

Referring back to FIGS. 1-2, as noted above, control console 540 is configured to receive the position data from magnetic sensor assemblies 534a-534d and, based thereupon, calculate the position and orientation of each magnet 510, 520, and output the calculated position and orientation data to the visualization application 620 of visualization system 600 to enable display of a 3D virtual environment of the real-time position of tracking magnets 510, 520, as detailed below.

Control console 540, more specifically, includes one or more processors 542 and a computer-readable storage medium 544 storing instructions to be executed by the processor(s) 542 to perform the above-noted functionality. The one or more processors 542 may be, for example, one or more digital signal processors (DSPs), general purpose microprocessors, application specific integrated circuits (ASICs), field programmable logic arrays (FPGAs), and/or other suitable integrated or discrete logic circuitry. Storage medium 544 may include any suitable computer-readable storage media, which corresponds to a tangible medium such as data storage media (e.g., RAM, ROM, EEPROM, flash memory, or any other medium that can be used to store desired program code in the form of instructions or data structures and that can be accessed by a computer). Control console 540 may be incorporated into one or more servers, tablets, smartphones, desktop computers, laptop computers, or the like.

Continuing with reference to FIGS. 1 and 2, visualization system 600, as noted above, includes visualization application 620 running on computer 610 and display 630 including user interface 632. Visualization system 600 may further include one or more systems 640, e.g., computers, servers, databases, applications, etc., with which visualization application 620 interfaces to, for example, receive data and/or allow importing, rendering, animating, and recording three-dimensional surface and/or volumetric models of patient specific tumors “T” (FIGS. 6A-7) and anatomy, e.g., breast “B” (FIG. 4); surgical instruments 20, 30, 40, and 50 (FIGS. 1, 6A-6C, 7, and 8, respectively) utilized during the procedure (e.g., based upon instrument information received); and/or visual aids, e.g., safe resection margins around the tumor “T” (FIGS. 6A-7), alignment axes, possible trajectories, etc. Such systems 640 may include, for example, identifier device 26 of surgical instrument 20 (FIG. 1), a Picture Archiving and Communication System (PACS), a Radiology Information System, an Electronic Medical Records System (EMR), a Laboratory Information System (LIS), etc.

Visualization system 600 provides an integrated real time 3D environment displayed on user interface 632 to facilitate navigation of a surgical instrument including tracking magnet 520 to a tissue structure including implanted tracking magnet 510. Visualization application 620, utilizing data from geomagnetic tracking system 500 and/or one or more of systems 640, may more specifically be configured to display the position and orientation of 3D virtual models and update the same in real-time based on the positioning of the tracked structures, e.g., tumor “T” via tracking magnet 510 and the surgical instrument via tracking magnet 520. Visualization application 620 may also change properties of the real time 3D environment such as color, opacity, triangulated surfaces, etc. dynamically according to feedback received; allow a surgeon to dynamically change the location and zoom of a virtual camera to show different views to the surgeon; and/or record the paths of tracked instruments and anatomy during surgery for documentation, training, and/or analysis purposes.

Utilizing data from geomagnetic tracking system 500 and/or one or more of systems 640, visualization application 620 may be configured to receive real time information on the location of a patient's anatomy, e.g., via an optical system. Visualization application 620 may also or alternatively electronically store or access anatomy or instrument identification information indicative of a particular tumor location or procedure assigned to the instrument (e.g., a biopsy vs. full tissue removal procedure). In embodiments, animation of the projected paths of movable portions of the instrument may be programmed into or accessed by visualization application 620 or help the surgeon predict the effect of the instrument. The path may be projected onto the user interface 632 in animation to enable the surgeon to confirm the path prior to actually working on tissue. Data from geomagnetic tracking system 500 and/or one or more of systems 600 can also be used in conjunction with augmented, extended, and/or virtual reality systems, to be used by surgeons during surgery and/or training and simulations.

Visualization application 620, utilizing data from geomagnetic tracking system 500 and/or one or more of systems 640, may incorporate tracking data from additional devices, e.g., an infrared tracking device, an optical tracking device, an acoustic tracking device, a radiation tracking device, a radar tracking device, a radiofrequency tracking device, an ultra-wide band tracking device, etc.

Visualization application 620, utilizing data from geomagnetic tracking system 500 and/or one or more of systems 640, may pre-register the patient's anatomy, including the location of a tumor “T” and the tumor margins. For example, patients' medical images (CT, MRI, US, etc.) can be processed before surgery to apply filters, segmentation, reconstruction, and other images processes to generate patient-specific 3D models of the tumors “T,” breasts “B,” and other anatomical structures. If there is no imaging suitable for 3D reconstruction, models of the tumor “T” can be created based on indirect measurements and parametric data provided by the surgeon (or other clinician). Moreover, based on the tumor's size/shape and some input that reflects medical judgment (e.g., required margin of healthy tissue), the system can calculate and create a 3D model to represent the size/shape of the tissue to be removed so as to fully contain the tumor “T” to be removed.

Computer 610 includes one or more processors 612 and a computer-readable storage medium 614 storing instructions to be executed by the processor(s) 612 to perform the above-noted functionality. The one or more processors 612 may be, for example, one or more digital signal processors (DSPs), general purpose microprocessors, application specific integrated circuits (ASICs), field programmable logic arrays (FPGAs), graphics processing units (GPUs), and/or other suitable integrated or discrete logic circuitry. Storage medium 614 may store visualization application 620 and/or may include any suitable computer-readable storage media, which corresponds to a tangible medium such as data storage media (e.g., RAM, ROM, EEPROM, flash memory, or any other medium that can be used to store desired program code in the form of instructions or data structures and that can be accessed by a computer). Alternatively, visualization application 620 may be run on any other suitable device and accessible via processor(s) 612. Computer 610 may be one or more servers, tablets, smartphones, desktop computers, laptop computers, or the like. Display 630 may be any suitable display capable of presenting a user interface 632.

Referring to FIGS. 6A-6C, 7, and 8, additional surgical instruments 30, 40, 50 configured for use with tracking and visualization system 10 are shown. FIGS. 6A-6C, for example, illustrate progressive use of a tissue removal device 30 to surround a tumor “T” having implanted therein tracking magnet 510. Tissue removal device 30 may be configured similarly as detailed in U.S. Patent Application Pub. No. 2017/0095286 by Vacha et al., the entire contents of which are hereby incorporated herein by reference, and generally includes a shaft 32 including tracking magnet 520 disposed at a distal end portion thereof, and an end effector extending distally from shaft 32 and including a pair of extendable arms 34 and an electrode assembly 36. Tracking and visualization provided by system 10, as appreciated, facilitates removal of tissue using device 30.

FIG. 7 illustrates a biopsy device 40 including a housing 42 and a needle 44 extending distally from housing 42. Tracking magnet 520 is disposed at a distal end portion of housing 42. Biopsy device 40 utilizes the tracking and visualization provided by system 10 to facilitate removal of a biopsy sample.

FIG. 8 illustrates an electrosurgical vessel sealer and divider 50 such as detailed in U.S. Pat. No. 7,156,846 to Dycus et al., the entire contents of which are hereby incorporated herein by reference, and generally includes a housing 52, a shaft 54 extending distally from housing 52, and an end effector assembly 56 including jaw members 58 disposed at the distal end of shaft 54. Tracking magnet 520 is disposed at a distal end portion of shaft 54.

Referring generally to FIGS. 1-8, in preparation for use with respect to a breast lumpectomy procedure, for example, tracking magnet 510 is implanted in the tumor “T” in the breast “B” as noted above or in any other suitable manner. A suitable surgical instrument incorporating tracking magnet 520 is selected or tracking magnet 520 is coupled to a suitable surgical instrument. Next, or prior to the above, sensor base assembly 530 is positioned as desired, e.g., about a patient's breast “B” with the breast “B” extending through opening 533 of base 532 such that magnetic sensor assemblies 534a-534d are arranged about the tumor “T” to be removed from within the breast “B.” In embodiments where necessary, the positions of magnetic sensor assemblies 534a-534d may be calibrated and stored in control console 540.

Control console 540 is then or prior thereto connected to visualization system 600, and the surgical instrument (or identifier device 26 (FIG. 1) associated therewith) is connected to control console 540 and/or visualization system 600 enabling visualization system 600 to recognizes the particular instrument information, e.g., the spatial location of the tip (or other relevant component/feature) of the instrument from tracking magnet 520.

In use, visualization system 600 provides the surgeon with visual 3D representations indicative of the tip (or other relevant component/feature) of the instrument relative to the patient's anatomy, e.g., a blade icon representing the distal tip of electrosurgical pencil 20 (FIG. 1) superimposed on a 3D model of the patient's breast “B” (FIG. 4) including tracking magnet 510 disposed within the tumor “T.” The visualization system 600 updates the representations as the surgeon maneuvers the instrument to bring the tip (or other relevant component/feature) of the instrument to the target site, thus facilitating removal of the tumor “T” (and tracking magnet 510 therewith) under visual guidance.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical tracking system, comprising:
a first tracking magnet;
a second tracking magnet; and
a sensor base assembly including a base defining an opening therethrough and at least three magnetic sensor assemblies disposed on the base and arranged about the opening, each of the at least three magnetic sensor assemblies configured to independently track each of the first and second tracking magnets in X, Y, and Z dimensions.

2. The surgical tracking system according to claim 1, further comprising:
a control console configured to receive tracking data from each of the at least three magnetic sensor assemblies and determine position and orientation information for each of the first and second tracking magnets based upon the received tracking data.

3. The surgical tracking system according to claim 1, wherein the sensor base assembly is configured for positioning such that at least a portion of tissue of interest extends through the opening of the base.

4. The surgical tracking system according to claim 1, wherein the first tracking magnet is configured for implantation into tissue of interest.

5. The surgical tracking system according to claim 4, wherein the sensor base assembly is configured for positioning such that at least a portion of the tissue of interest extends through the opening of the base.

6. The surgical tracking system according to claim 1, wherein the second tracking magnet is coupled to a surgical instrument.

7. The surgical tracking system according to claim 6, wherein the second tracking magnet is incorporated into the surgical instrument.

8. The surgical tracking system according to claim 1, wherein the base is adjustable to adjust at least one dimension of the opening of the base.

9. The surgical tracking system according to claim 1, wherein the at least three magnetic sensor assemblies includes at least four magnetic sensor assemblies radially arranged about the base.

10. The surgical tracking system according to claim 1, wherein the at least three magnetic sensor assemblies includes at least eight magnetic sensor assemblies radially arranged about the base.

11. A surgical visualization and tracking system, comprising:
a surgical tracking system, including:
a first tracking magnet;
a second tracking magnet;
a sensor base assembly including at least three magnetic sensor assemblies, each of the at least three magnetic sensor assemblies configured to independently track each of the first and second tracking magnets in X, Y, and Z dimensions; and
a control console configured to receive tracking data from each of the at least three magnetic sensor assemblies and determine position and orientation information for each of the first and second tracking magnets based upon the received tracking data; and
a visualization system configured to receive the position and orientation information from the control console and represent the position and orientation information for each of the first and second tracking magnets on a displayed 3D model of tissue.

12. The surgical visualization and tracking system according to claim 11, wherein the sensor base assembly further includes a base defining an opening therethrough, and wherein the at least three magnetic sensor assemblies are disposed on the base and arranged about the opening.

13. The surgical visualization and tracking system according to claim 12, wherein the base is configured for positioning such that at least a portion of tissue of interest extends through the opening of the base.

14. The surgical visualization and tracking system according to claim 11, wherein:
the first tracking magnet is configured for implantation into tissue of interest; and
the second tracking magnet is associated with a surgical instrument.

15. The surgical visualization and tracking system according to claim 11, wherein the at least three magnetic sensor assemblies includes four magnetic sensor assemblies radially arranged.

16. The surgical visualization and tracking system according to claim 11, wherein the visualization system is further configured to receive instrument information regarding a surgical instrument with which the second tracking magnet is associated and wherein representing the position and orientation information for the second tracking magnet on the displayed 3D model of tissue includes representing at least a portion of the surgical instrument on the displayed 3D model of tissue.

17. The surgical visualization and tracking system according to claim 16, wherein the instrument information includes information indicating a distance from the second tracking magnet to a feature of the surgical instrument.

18. The surgical visualization and tracking system according to claim 11, wherein the represented position and orientation information is updated in real time.

19. The surgical visualization and tracking system according to claim 11, wherein the visualization system includes a non-transitory computer-readable storage medium storing a visualization application that, when executed by a processor, receives the position and orientation information from the control console and represents the position and orientation information for each of the first and second tracking magnets on the displayed 3D model of tissue.

20. The surgical visualization and tracking system according to claim 19, wherein the visualization system further comprises a display including a user interface, and wherein the visualization application, when executed by the processor, outputs the representation and the displayed 3D model of tissue for display on the user interface of the display.
